(19) United States
(12) Patent Application Publication
(10) Pub. No.: US 2009/0041618 A1
(43) Pub. Date: Feb. 12, 2009

(54) Reusable porous metal filter

(75) Inventors: Marcia A. Frieze, Alpine, NJ (US); Allan S. Frieze, Alpine, NJ (US)

Correspondence Address: Irving M. Fishman, Suite 1422, North Tower, 89 Headquarters Plaza, Morristown, NJ, 07960, US

(73) Assignee: Case Medical Inc.

(21) Appl. No.: 12/217,858

(22) Filed: Jul. 9, 2008

Related U.S. Application Data
(60) Provisional application No. 60/954,164, filed on Aug. 6, 2007.

(51) Int. Cl. A61L 2/14; B01D 39/10; A61L 2/00; A61L 2/20; A61L 2/07

(52) U.S. Cl. 422/22; 210/500.25; 422/1; 422/26; 422/28

(57) ABSTRACT

A porous metal filter for use in conjunction with a sterilization container where the porous metal filter is fabricated from metal powder or metal pellets having an average particle size of at least greater than 0.2 microns and a thickness of at least about 15 mils (0.015″, about 381 microns) is disclosed. The filter is compatible with all current sterilization environments and all current sterilization procedures.

### CROSS-REFERENCE TO RELATED APPLICATIONS

This application claims priority of U.S. Provisional Application Ser. No. 60/954,164, filed Jul. 11, 2007, which is incorporated herein in its entirety by reference.

### STATEMENT REGARDING FEDERALLY SPONSORED RESEARCH OR DEVELOPMENT

Not Applicable

### FIELD OF THE INVENTION

The present invention relates to porous metal plates and their use as reusable filters in sterilization containers.

### BACKGROUND OF THE INVENTION

Sterilization containers are described in the art in a number of contexts. The inventors issued U.S. Pat. No. 5,968,459 and its family members (all incorporated herein by reference in their entirety) (inclusive of U.S. Pat. No. 6,589,477 U.S. Pat. No. 6,468,482, US 2005-0238530, US 2003-0118491, and US 2002-0136679 among others) describe sterilization containers using filters of various kinds where the filter is either reusable or disposable and usually held in place by a filter retention plate. Specific filter materials mentioned are paper, TEFLON, porous stainless steel, polysulfone, and hydrophobic materials such as GORTEX. (See column 2, line 65-column 3, line 1 of U.S. Pat. No. 5,968,459.) However, other than a bland mentioning of porous stainless steel, the entire family of patents and applications are silent as to any of the porous stainless steel properties or specifications needed.

Porous stainless steel is known as is described in U.S. Pat. No. 4,217,415 (enzyme immobilization on or in the pores of a porous substrate, which substrate can be porous stainless steel); U.S. Pat. No. 4,927,637 and U.S. Pat. No. 4,716,044 (forced pressure filtration through membrane filters on a porous support, which porous support can be porous stainless steel); U.S. Pat. No. 6,746,580, U.S. Pat. No. 6,733,638 and U.S. Pat. No. 6,461,487 (use in electrodes); U.S. Pat. No. 6,575,308 (general use as a filter among a number of alternatives, especially ceramics); U.S. Pat. No. 6,719,147 (supported mesoporous carbon membranes where the carbon membrane is on or in the support and the support can be porous stainless steel); U.S. Pat. No. 5,554,283; U.S. Pat. No. 5,543,002, and U.S. Pat. No. 5,531,848 (general mention of porous stainless steel); and U.S. Pat. No. 5,431,829 (discussing the disadvantages of porous stainless steel as a filtration member in the context of fluid filtration), all of which are incorporated herein by reference. In particular, U.S. Pat. No. 5,431,829 discusses the disadvantages of porous stainless steel of excessive cost, rapid clogging resulting is short cycle times, and difficulty in disposal.

One of the significant problems of a filter member in a sterilization container for medical instruments is that the container may be used in a number of different types of sterilization, such as steam, gas, gas plasma, etc and may be used in different types of operating conditions such as terminal sterilization or flash sterilization, gravity displacement or pre-vacuum. Each of these has differing requirements and locating a reusable material that is suitable in all of these environments and conditions has been sought after for some time.

### OBJECTS OF THE INVENTION

It is therefore an object of the invention to provide a filtration member for use in multiple sterilization environments and procedures, inclusive of hydrogen peroxide gas plasma sterilization.

Another object of the invention is to provide a sterilization case for sterilizing materials having a filtration member which is compatible with a number of different sterilization environments and techniques.

Yet another object of the invention is to provide a sterilization method which includes sterilization of materials in a sterilization case that contains as a filtration member a filter that is compatible with multiple sterilization environments, inclusive of hydrogen peroxide gas plasma sterilization, and a variety of sterilization procedures.

Still another object of the invention is to provide a sterilization container filter which is a porous metal filter which is suitable for use in multiple sterilization environments, inclusive of hydrogen peroxide gas plasma sterilization, and a variety of sterilization techniques.

Another object of the invention is to provide a reusable filter compatible with each of terminal and flash cycle sterilization and is thermoconductive, non-insulating, non-breakable and avoids strike through.

Yet further objects of the invention will be appreciated by those of ordinary skill in the art.

### BRIEF SUMMARY OF THE INVENTION

These and other objects of the invention can be achieved by a porous metal filter for use in conjunction with a sterilization container where the porous metal filter is fabricated from metal powder or metal fibers, or metal pellets having an average particle size of at least greater than 0.2 microns and less than about 50 microns; and the plate has a thickness of at least about 15 mils (about 0.015″, about 381 microns) and generally less than about 50 mils (about 0.05″, about 1.27 mm). The filter is compatible with all current sterilization environments and all current sterilization procedures and can be interchanged for other filters (whether or not porous metal) having similar mating components or used in conjunction with an adapter to make such interchange possible. The porous metal filters of the invention can also be interchanged for other similar porous metal filters to allow for the sterilization container to be rapidly reused while the porous filter undergoes a cleaning operation. The porous metal filter plate has the same mating features for interchangeability as the previously cleared devices.

### BRIEF DESCRIPTION OF THE SEVERAL VIEWS OF THE DRAWING

Not Applicable

### DETAILED DESCRIPTION OF THE INVENTION

The present invention is an improvement in the particular filter material and the limitations thereon used in a gas plasma sterilization compatible container and gas plasma sterilization technique compatible techniques. In particular, the invention relates to the use of a porous metal plate as a filtration member for use in conjunction with a sterilization container in a sterilization procedure. The porous metal plate is generally manufactured from powdered or fibrous or pelleted metal, pressed into a flat sheet of desired thickness (that can be machined to desired sizes and shapes) or molded into the desired size, shape, and thickness. The pressed sheet or molded material is then typically sintered. Those of ordinary skill in the art will be able to manufacture such plates once given the limitations of the plates set forth in this specification using known techniques and procedures.

The metal is one that has a high thermoconductivity, such as titanium, aluminum, carbon, copper, gold, silver, tungsten, alloys thereof, stainless steel, and brass, preferably stainless steel. Preferably, the metal powder used to prepare the porous metal plate has an average particle size of at least greater than 0.2 micron, more preferably at least about 0.25 micron, still more preferably at least about 0.3 micron, even more preferably at least about 0.4 micron, most preferably at least about 0.5 micron. The particle size used should generally be less than about 1 micron, preferably not in excess of about 0.9 micron, still more preferably not in excess of about 0.8 micron, more preferably not in excess of 0.75 micron, yet more preferably not in excess of about 0.6 micron, most preferably about 0.5 micron. All ranges obtained by selecting one of these minimums and one of these maximums are deemed to be specifically disclosed herein. Highly preferably, the particle size of the powder or fibers or pellets used to manufacture the porous metal plate is from about 0.4 micron to about 0.6 micron, most preferably about 0.5 micron. The plate thickness is generally suitable from plates that are at least greater than about 15 mils (about 0.015″, about 381 microns) thick, more preferably at least about 16 mils (about 0.016″, about 406.4 microns); more preferably at least about 17 mils (about 0.017″, about 431.8 microns); even more preferably at least about 18 mils (about 0.018″, about 457.2 microns); yet more preferably at least about 19 mils (about 482.6 microns), still even more preferably at least about 20 mils (about 0.02″, about 508 microns); further more preferably at least about 25 mils (about 0.025″, about 635 microns); most preferably at least about 30 mils (about 0.03″, about 762 microns). The plate maximum thickness is less important, and really only limited by a desire to keep the product weight as low as possible. Nonetheless, for this reason, the maximum thickness should be kept to generally not more than about 75 mils (about 0.075″, about 1.91 mm); still more preferably not more than about 50 mils (about 0.05″, about 1.27 mm); still more preferably not more than about 48 mils (about 0.048″, about 1.22 mm); even more preferably not more than about 46 mils (about 0.046″, about 1.1684 mm); still even more preferably not more than about 45 mils (about 0.045″, about 1.1176 mm); yet more preferably about 40 mils (about 0.04″, about 1.016 mm); still even more preferably not more than about 35 mils (about 0.035″, 889 micron). All ranges obtained by selecting any of the above minimums and any of the above maximums for the plate thickness are deemed expressly disclosed herein.

Surprising advantages of the porous metal filter described above is that it can be used with a sterilization case in any sterilization environment, including, but not limited to, steam, gas, gas plasma (particularly hydrogen peroxide gas plasma) and can be used in any sterilization procedure such as, without limitation, gravity displacement, pre-vacuum, and other techniques, and can be used in any time frame, whether terminal sterilization or flash sterilization. Thus, the present invention allows for the elimination of disposable filter thus avoiding, waste, eliminating reprocessing due to tears or punctures and for the elimination of a valve in steam sterilization (thereby making the container simpler and more reliable as one need not be concerned with the valve functioning properly); allows for fewer types of parts needing to be stored (a single type of filter can be used in all cases), an advantage for standardization; and allows for reduction in errors in placing the wrong type of filter onto a sterilization unit (i.e., one that is not compatible with the sterilization medium or technique being used). Thus, the number and type of filter units that need be maintained in storage for ready availability for use can be substantially reduced (and valve units can be eliminated from storage) resulting in substantial reductions in storage requirements. Furthermore, the porous metal filters may be cleaned and are reusable so that (a) costs are reduced and (b) environmental impact of disposable medical waste product is reduced. Furthermore the metal filter is thermoconductive, non-breakable and easily decontaminated for reuse as opposed to ceramic or synthetic reusable filters that are non-thermoconductive, and either breakable or subject to wear/punctures.

Another surprising advantage of the present invention is that when maintaining the parameters of minimum particle size from which the porous metal plates are obtained, filtered flash sterilization is possible. For example, paper and polypropylene disposable filters in a gravity displacement steam sterilization require extended exposure times of 30 to 40 minutes. With the porous metal plate of the present invention, the exposure time for effective sterilization in gravity displacement steam sterilization drops to about 3-5 minutes. This is of extreme importance in the medical instrument sterilization setting where an instrument may need to be sterilized quickly during the course of an ongoing medical procedure or where there is a large turnover of patients and instruments in use with one patient are needed for use with the another patient on an urgent basis. The filtered sterilization containers using the invention porous metal plate filter also performs well in pre-vacuum steam, in gas plasma, and hydrogen peroxide plasma in both standard and advanced cycles. Substantially similar results can be expected from use thereof with ethylene oxide, ozone, hydrogen peroxide, and carbon dioxide sterilization media.

The thickness of the porous metal filter is described above. Other than that thickness, the size and shape of the filter is essentially dictated only by the fact that when mounted on the appropriate portion of the sterilization container, that it cover the hole through which the sterilization media traverses from an exterior to interior portion of the case or from an interior portion to exterior portion of the case. This is so that the filter prevents microorganisms and other debris from getting into the sterilization container between the sterilization procedure and the opening of the sterilization container for removal of the instruments that have been sterilized. Larger cross sectional areas are permitted, but make the plate unnecessarily heavy and more expensive.

The porous metal plate filter of the present invention can further be formed with appropriate mating components to easily attach the porous metal filter directly to mating components on the sterilization container so that no retention plate is needed to hold the porous metal plate onto the sterilization container. Alternatively, it can be used in conjunction with a retention plate or suitable adapter so as to allow suitable mating therewith. The attachment of the filter to the sterilization container is in accordance with the disclosure of such retention plates and mating components as set forth in the Inventor's patent U.S. Pat. No. 5,968,459 and its progeny (all incorporated herein by reference) or other suitable attachment means as may be known in the art.

Still further, in certain sterilization containers, such as one by Riley Medical (now Symmetry Medical), a plastic case is used with a pressure sensitive valve to rapidly sterilize loads in a steam flash cycle. The system contains a plug that needs to be burped or manually removed to express the steam and relieve the pressure. In addition, the device's pressure sensitive valve system requires daily calibration. It appears that this system also has a side vent that appears to be porous metal that is permanently affixed to the plastic container. It is not removable nor is it recommended for cleaning and as a result can clog with sediment, biomatter or hard water deposits. Thus, this side vent is not a replacement of and does not operate in the manner of the primary inlet for sterilization purposes.

In addition to the above advantages, it should be noted that as a metal plate, the filter, in operation, is not subject to tears and/or punctures due to strike through either externally by tampering, or internally due to punctures from medical instruments placed in the container for sterilization. This is a substantial advantage over the polypropylene filters (which are compatible with the various sterilization environments) but are disposable and subject to tear. The invention porous metal plate is puncture proof (thereby avoiding the necessity of concern with tear and thereby avoiding the issue of offset to avoid strikethrough (discussed in US 2002-136679 and US 2003-0118491). Sterilization containers from Monarch (formerly Sterion, now Steris) (indicated only for steam sterilization) utilize a ceramic filter which is subject to cracking, while the present invention filters do not crack.

In addition to the above, the porous metal plate filters of the present invention, if desired, can be permanently affixed to the sterilization container, although the removable and interchangeable arrangement is preferred. When the porous metal plate filter of the invention is desired to be permanently affixed to the sterilization container, the container part may still be used in any sterilization context, but now, the entire lid or bottom of the container to which it is attached needs to be subjected to the cleaning operation of the porous metal filter and the advantages of interchangeability are lost. Still, the advantage of a permanently affixed porous metal filter is that a container lid need only be matched with a container bottom and one need not bother with the operation of attaching the porous metal filter.

We claim:

1. A reusable porous metal plate for use as a filter in a sterilization container system comprising a porous metal plate said porous metal plate being fabricated from a metal powder or metal pellets having an average particle size of or metal fiber having a fiber thickness of at least greater than about 0.2 micron.

2. The porous metal plate of claim 1 being fabricated from a metal powder or metal pellets having an average particle size of or metal fiber having a fiber thickness of less than about 50 microns.

3. The porous metal plate of claim 1 having a plate thickness of at least about 15 mil (about 0.015″, about 381 micron).

4. The porous metal plate of claim 1 having a plate thickness of less than about 50 mil (about 0.05″, about 1.27 mm).

5. The porous metal plate of claim 1 wherein said porous metal plate comprises a metal selected from the group consisting of those with a high thermoconductivity.

6. The porous metal plate of claim 1 wherein said porous metal plate comprises a metal selected from the group consisting of titanium, aluminum, copper, carbon, gold, alloys thereof, stainless steel, and brass.

7. The porous metal plate of claim 1 wherein said porous metal plate comprises stainless steel.

8. A sterilization container having as a filter member the porous metal plate of claim 1.

9. The sterilization container of claim 8 wherein said filter member is removable from said sterilization container.

10. The sterilization container of claim 9 wherein said filter member is permanently affixed to said sterilization container.

11. A method of sterilization of an article comprising placing said article into a sterilization container of claim 9 and subjecting said container with said article therein to a sterilization operation.

12. The method of claim 1 wherein said sterilization operation is a sterilization procedure selected from steam sterilization, gas sterilization and gas plasma sterilization.

13. The method of claim 12 wherein said sterilization operation is a sterilization process selected from pre-vacuum sterilization, and gravity displacement steam sterilization.

14. The method of claim 12 wherein said sterilization operation is conducted under terminal sterilization or flash sterilization conditions.

15. A method of using a porous metal plate (pmp) as a filter element in conjunction with an anodized aluminum sterilization container, said pmp being fabricated from a metal powder or metal fibers or metal pellets having an average particle size of at least about 0.2 micron to less than about 1 micron, said pmp, when in use in a sterilization operation, operating to allow passage of sterilant into and/or out of said container.

16. The method of claim 15 wherein said pmp has a thickness of at least about 15 mils (0.015″, about 381 micron) to less than about 50 mils (0.05″, about 1.27 mm).

17. The method of claim 15 wherein said pmp is interchangeable with filter materials that are (a) not porous metal and/or (b) that are porous metal but not being fabricated from a metal powder or metal pellets having an average particle size of or from metal fiber having a fiber thickness in the range of at least about 0.2 micron up to about 1 micron and/or that are porous metal but have a plate thickness not in the range of about 15 mils (0.015″, about 381 microns) to about 50 mils (0.05″, about 1.27 mm).
